(19) United States
(12) Patent Application Publication
(10) Pub. No.: US 2009/0038551 A1
(43) Pub. Date: Feb. 12, 2009

(54) MILK PRODUCTION IN A CONTROLLED ENVIRONMENT

(76) Inventors: Jon Baker, Sandy, UT (US); Mark Lindsey, Salt Lake City, UT (US)

Correspondence Address: OGILVIE LAW FIRM, 1320 EAST LAIRD AVENUE, SALT LAKE CITY, UT, 84105, US

(21) Appl. No.: 12/184,434

(22) Filed: Aug. 1, 2008

Related U.S. Application Data
(60) Provisional application No. 60/954,573, filed on Aug. 8, 2007.

(51) Int. Cl. A01J 5/00

(52) U.S. Cl. 119/14.02

(57) ABSTRACT

A milk-producing animal is provided with decontaminated air, decontaminated water, and decontaminated feed inside a hermetically sealed decontaminated facility. Uncontaminated milk is then obtained from the animal within the facility. The animal is decontaminated and vaccinated before being placed in the facility. Human personnel decontamination, a mandatory airlock, and/or automated livestock equipment are also utilized to limit contamination of the hermetically sealed facility.

### RELATED APPLICATIONS

The present application claims priority to, and incorporates by reference, U.S. provisional patent application Ser. No. 60/954,573 filed Aug. 8, 2007.

### BACKGROUND

Milk is used commercially in a variety of consumer goods, including cheese, yogurt, ice cream, and other dairy goods. Milk is often pasteurized before it is processed into dairy goods. In some cases, unpasteurized milk is used when making cheese or other dairy goods. Even when milk is pasteurized, however, some bacteria may remain.

More generally, pathogens in milk can pose serious health risks. Accordingly, pasteurization, filtration, and other processes are often used to treat milk, in order to inactivate pathogens and/or to remove them from the milk. Milk may be rapidly cooled to reduce bacterial growth. Tests are often performed on dairy farms and in milk processing facilities to detect pathogens. Unacceptable milk is discarded. Cows infected with mastitis are treated, and they may be culled from the dairy herd if their infection does not respond adequately to treatment.

### SUMMARY

In some embodiments, a dairy cow or other milk-producing animal is provided with decontaminated air, decontaminated water, and decontaminated feed inside a hermetically sealed decontaminated facility. Uncontaminated milk is then obtained from the animal within the facility. The animal is decontaminated and vaccinated if necessary before being placed in the facility. Human personnel are required to put on protective garments and/or are decontaminated before entering the facility, to limit human-animal contact that may contaminate the animal. In some embodiments, a mandatory airlock and/or extensive automation are utilized to limit contamination of the hermetically sealed facility. In some embodiments, a high grade milk is produced by maintaining within a hermetically sealed facility no more than a maximum specified contaminant level of pathogens such as tuberculosis, brucellosis, Johne's disease, salmonella, foot-in-mouth disease, or contagious mastitis.

The examples given are merely illustrative. This Summary is not intended to identify key features or essential features of the claimed subject matter, nor is it intended to be used to limit the scope of the claimed subject matter. Rather, this Summary is provided to introduce—in a simplified form—some concepts that are further described below in the Detailed Description. The innovation is defined with claims, and to the extent this Summary conflicts with the claims, the claims should prevail.

### DESCRIPTION OF THE DRAWINGS

A more particular description will be given with reference to the attached drawings. These drawings only illustrate selected aspects and thus do not fully determine coverage or scope.

FIG. 1 is a block diagram illustrating a livestock facility in an operating environment;

FIG. 2 is a block diagram further illustrating a livestock facility, including some details about livestock equipment and decontaminated life-sustaining materials;

FIG. 3 is a block diagram further illustrating a livestock facility, including some details about an airlock;

FIG. 4 is a flow chart illustrating steps of some method embodiments, including steps for providing decontaminated life-sustaining materials to livestock; and

FIG. 5 is a flow chart illustrating steps of some method embodiments, including decontamination steps for a livestock facility, livestock, and people.

### DETAILED DESCRIPTION

Overview

Despite the use of pasteurization and other familiar measures, improvements can be made in the purity of milk. New approaches to improving milk purity are provided herein, using for example a hermetically sealed facility in which dairy cows are provided with decontaminated feed, filtered air, and purified water. Approaches to improve the purity of other animal husbandry products are also described here.

Reference will be made to exemplary embodiments such as those illustrated in the drawings, and specific language will be used herein to describe the same. But alterations and further modifications of the features illustrated herein, and additional applications of the principles illustrated herein, which would occur to one skilled in the relevant art(s) and having possession of this disclosure, should be considered within the scope of the claims.

The meaning of terms is clarified in this disclosure, so the claims should be read with careful attention to these clarifications. Specific examples are given, but those of skill in the relevant art(s) will understand that other examples may also fall within the meaning of the terms used, and within the scope of one or more claims. Terms do not necessarily have the same meaning here that they have in general usage, in animal husbandry generally, in the usage of a particular industry, or in a particular dictionary, set of dictionaries, or other publication. Reference numerals may be used with various phrasings, to help show the breadth of a term. Omission of a reference numeral from a given piece of text does not necessarily mean that the content of a Figure is not being discussed by the text. The inventors assert and exercise their right to their own lexicography. Terms may be defined, either explicitly with quotation marks or implicitly, here in the Detailed Description and/or elsewhere in the application file.

As used herein, a “hermetically sealed facility” is an enclosure which is either airtight or equipped with positive air pressure, or both. In some embodiments, a hermetically sealed facility has an airlock, not merely a door. The facility enclosure may include a barn or other building, or one or more rooms, domes, or other chambers, for example.

A “hermetically sealed decontaminated facility” is a hermetically sealed facility having contained air and interior surfaces that qualify as decontaminated. Air, water, feed, a surface, an animal, or a person, for example, may qualify as decontaminated by virtue of (i) meeting a specified relative contaminant level, (ii) meeting a specified absolute contaminant level, and/or (iii) having undergone a specified decontamination procedure. A hermetically sealed facility may qualify as “decontaminated” in connection with milk production and/or other livestock-based activities without necessarily meeting the standards of cleanliness that are required of clean facilities intended for other purposes, such as semiconductor fabrication foundries or hospital operating rooms.

Livestock feed within a hermetically sealed facility may qualify as “decontaminated feed” by meeting a specified relative contaminant level, by meeting a specified absolute contaminant level, and/or by undergoing a specified decontamination procedure.

As an example of a specified relative contaminant level, in one embodiment feed qualifies as decontaminated feed if it contains no more than 5% the level of fecal matter (e.g., bird and rodent droppings) that is typically found in the same type of feed outside the hermetically sealed facility. As another example of a specified relative contaminant level, in one embodiment feed qualifies as decontaminated feed if it contains no more than 15% the level of foreign matter that is typically found in the same type of feed outside the hermetically sealed facility. “Foreign matter” in feed includes bird droppings, rodent droppings, other fecal matter, soil, and animal carcass or other animal remains, for example. As another example of a specified relative contaminant level, in one embodiment feed qualifies as decontaminated feed if it contains no contaminant at more than 10% of an action level established by the United States Food and Drug Administration for the contaminant.

As an example of a specified absolute contaminant level, in one embodiment feed qualifies as decontaminated feed if it contains no more than 1 ppb of Aflatoxin. As another example of a specified absolute contaminant level, in one embodiment feed qualifies as decontaminated feed if it contains Deoxynivalenol at no more than 2 ppm in total ration and 4 ppm in grain.

As an example of a specified decontamination procedure, in one embodiment feed qualifies as decontaminated feed if it has been treated to inhibit mold growth using any anti-mold procedure described in an issued patent. As another example of a specified decontamination procedure, in one embodiment feed qualifies as decontaminated feed if it was produced from decontaminated stock within a hermetically sealed decontaminated facility and then transported without significant contamination to a milk-producing animal in the same or another hermetically sealed decontaminated facility.

As used here, “decontamination” and “decontaminated” indicate relative or absolute freedom from contaminants as indicated, regardless of whether the lack of contaminants was achieved by removing contaminants, by avoiding contaminants, and/or by rendering contaminants biologically inert. Contaminants are “removed” from material if they are physically separated from the material and/or if they are rendered biologically inert. “Biologically inert” material is inorganic material which does not readily host microbes. For example, plastic is biologically inert, while hay is not. “Uncontaminated” is used interchangeably with “decontaminated”.

“Contaminants” are items that carry, cause, or contribute directly to disease. Some examples of contaminants include unwanted microbes (certain bacteria are desirable in livestock), radionuclides, unclean airborne particulates, pathogens and other disease vectors, and unclean livestock manure and other unclean fecal matter. Excess heat and excess humidity are also contaminants, to the extent they contribute to poor health or disease in livestock.

“Pollutants” are by-products of animal husbandry which may degrade the environment inside or outside an animal husbandry facility. Some examples of pollutants include exhaust gases produced by livestock, and in particular the odors and volatile organic compounds within such exhaust gases.

“Life-sustaining material” means air, water, and/or livestock feed.

A “milk-producing animal” is a non-human mammal which is capable of producing milk, e.g., to nourish its offspring and/or for use by people. Some examples of milk-producing animals are cows, sheep, goats, horses, donkeys, bison, buffalo, antelope, elk, deer, llamas, canines, felines, pigs, chimpanzees, rabbits, and camels, among others.

A “cow” is a female of the family Bovidae, including for example, buffalo, bison, wild and domesticated cattle, sheep, goats, and water buffalo.

“Livestock” means fully or partially domesticated animals, and captive wild land and air animals. Milk-producing animals and poultry are livestock, for example. Livestock does not include microbes, fish, or humans, for example.

“Livestock products” are milk, meat, organs, hormones, leather, wool, and other items obtained from livestock bodies.

“Animal husbandry” means activities involving livestock, such as feeding livestock, raising livestock, providing care for livestock, and obtaining livestock products, for example. The terms “animal husbandry facility” and “livestock facility” are used interchangeably herein.

“Livestock equipment” means livestock milking equipment, livestock feeding equipment, livestock watering equipment, livestock manure removal equipment, livestock bedding, and/or livestock exercise equipment.

A “microbe” is a microscopic organism. Examples of microbes include bacteria, viruses, molds, fungi, algae, and protozoa, among others.

A “protective garment” is a garment designed to reduce or prevent transfer of microbes. Examples of protective garments include face masks, hair coverings, latex gloves, sterile suits, and the like. Examples of protective garments may be found in hospitals, semiconductor clean rooms, and laboratories conducting research with microbes.

“Nutraceutical” milk or other livestock products are products which have, or likely have, a physiological benefit beyond simple nutritional value, such as improved resistance to disease or improved organ system performance. The physiological benefit may be demonstrated or otherwise scientifically established, or it may be considered a likely result of reduced contamination in the product.

Operating Environments

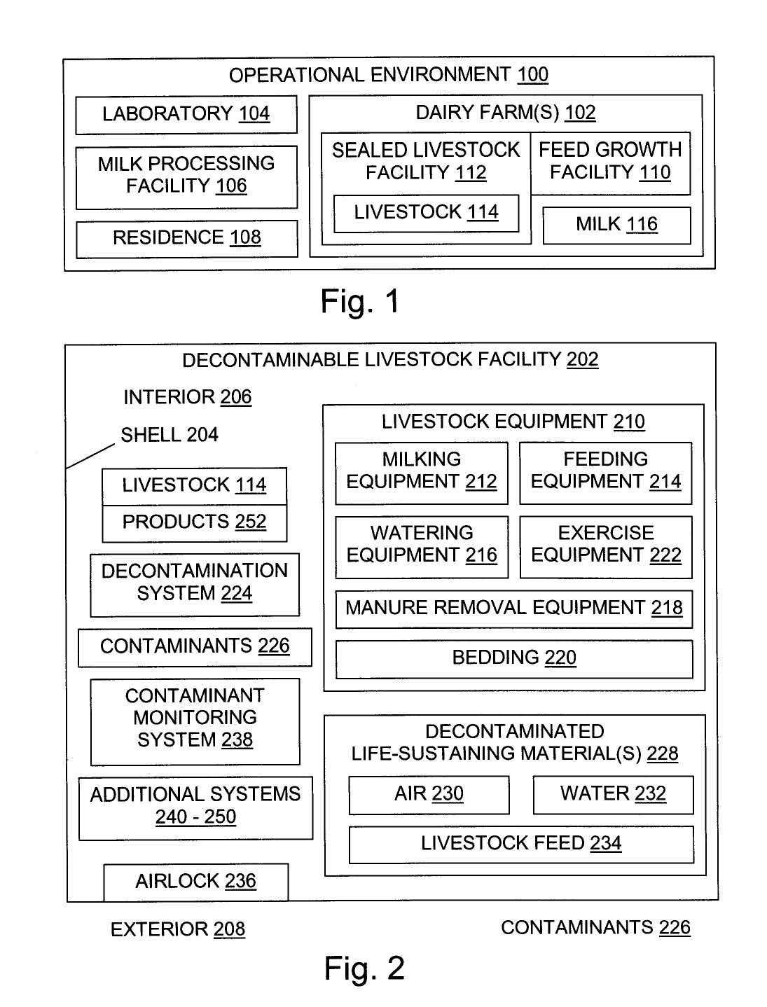

With reference to FIG. 1, an operating environment 100 for an embodiment may include, for instance, a commercial dairy farm 102, an experimental dairy farm 102 with an on-site laboratory 104, a dairy farm 102 integrated with an on-site milk processing facility 106, a dairy farm 102 integrated with an on-site human residence 108, a dairy farm 102 integrated with an on-site hydroponic feed growth facility 110, and/or a hermetically sealed livestock products facility 112 for producing livestock products other than milk or livestock products in addition to milk. Livestock 114 within the facility 112 are protected from pathogens and contaminants by the hermetic seal, by decontamination procedures and tools, by access controls, and/or by other measures described herein. Accordingly, milk 116 and other livestock products produced within the facility may be substantially more pure than would be the case without the decontamination tools and techniques described herein.

Sometimes a choice must be made between an embodiment described here and a previous approach. For example, a dairy cow cannot be enclosed in a hermetically sealed facility and at the same time be a free range cow. But in other situations an approach presented here is not incompatible with previously known aspects of animal husbandry, such as vaccination or exposure to natural lighting, for example. Accordingly, many of the present embodiments include familiar animal husbandry practices within their operating environments, suitably adapted for use with the embodiment.

Livestock Facilities

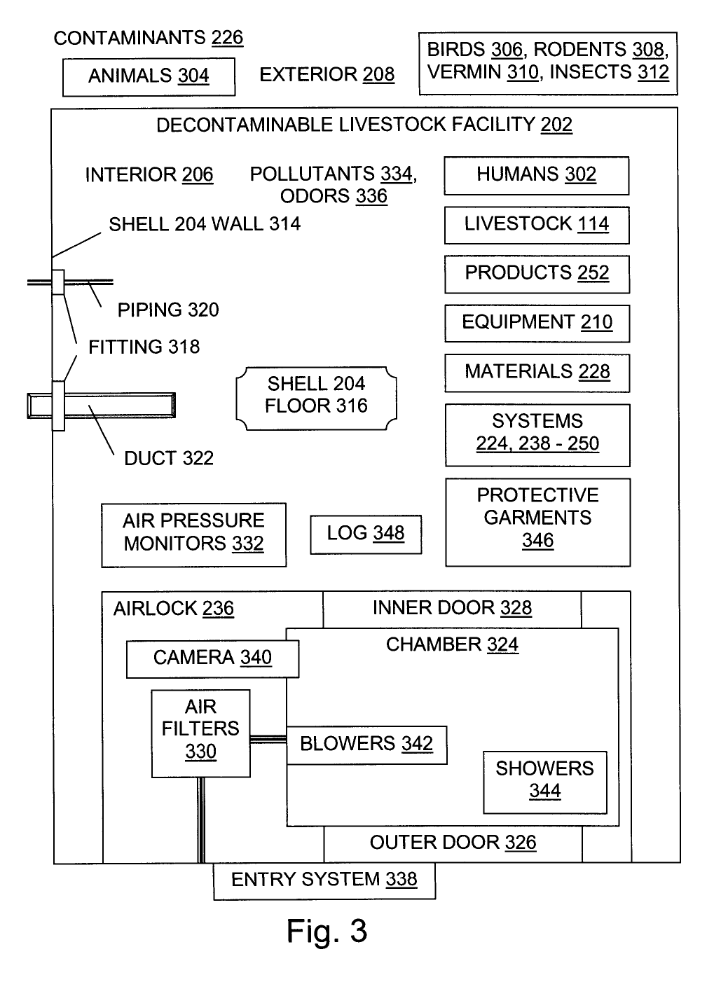

Referring now to FIGS. 1 through 3, some embodiments provide a decontaminable livestock facility 202 having a hermetically sealed shell 204 defining an enclosed interior 206 and an exterior 208. A hermetically sealed livestock products facility 112 for producing livestock products other than milk or producing livestock products in addition to milk is one of many possible examples of a facility 202.

Livestock equipment 210 is situated and operable within the facility interior 206. Some examples of livestock equipment 210 include livestock milking equipment 212, livestock feeding equipment 214, livestock watering equipment 216, livestock manure removal equipment 218, livestock bedding 220, and livestock exercise equipment 222.

A decontamination system 224 is physically integrated with the shell 204 and is operable to remove contaminants 226 from life-sustaining material 228 that is directed toward the interior 206. That is, the decontamination system 224 removes contaminants from at least one of the following life-sustaining materials: air 230, water 232, livestock feed 234.

Some embodiments have at least one airlock 236 and use decontamination procedures to help reduce contamination of the interior 206. A contaminant monitoring system 238 is operable to monitor contaminant levels within the interior 206 of some embodiments. A positive air pressure system 240 helps reduce contamination in some embodiments. Some embodiments also include a temperature control system 242, a humidity control system 244, a burner system 246, an electric power generation system 248, and/or a water treatment system 250.

Uncontaminated milk and/or other livestock products 252 are obtained from livestock 114 within the facility. Uncontaminated milk and/or other livestock products 252 may also be obtained from livestock which were housed within the facility 202 at some time and then transported without unacceptable contamination occurring during transport.

The shell 204 helps provide a barrier between (i) sources of possible infection and other contaminants 226, and (ii) a dairy herd and/or other livestock 114 housed in the facility. In a given embodiment, the facility 202 is designed to impose and maintain an effective permanent, semi-permanent, or temporary barrier between the livestock 114 and sources of possible infection. Possible sources of infection which may be barred from the facility or decontaminated before entry into the facility's interior include air 230, water 232, feed 234, humans 302, other cows or animals 304 (livestock or not) from the exterior 208, birds 306, rodents 308, vermin 310, and flies and other flying or airborne insects 312, for example.

In some embodiments the materials and construction used in the facility are “penetration-resistant”. Penetration-resistant materials are highly resistant to (i) penetration by hail, ice, snow, rainwater, and windborne objects; (ii) ultraviolet and infrared light from the sun, and other radiation degradation effects; (iii) gnawing by rodents; (iv) collisions, pecking, and other attacks by birds; (v) attacks by airborne and burrowing insects; (vi) penetration by vermin generally, even through extremely small cracks such as cracks the width of an average local ant or termite; and/or (vii) penetration by microscopic viruses, spores, bacteria, and other microbes.

In a given embodiment, suitable penetration-resistant materials for a shell 204 include high-tech scrims and/or other moderately high to high tensile strength woven synthetic fabrics, such as a woven polyethylene fabric, and materials with similar barrier properties. Other penetration-resistant materials may also be used, including for example metal, plastic, and glass. Unassisted and untreated wood of the varieties used in conventional barns is not penetration-resistant. The shell material is highly resistant to gnawing and tearing, to prevent rodents and other vermin from gaining access to the interior of the facility. In some embodiments, the pore space in external fabric and other shell material is small enough to prevent a virus from passing through the shell. Shell materials may be coated with vapor barriers, resins, insecticides, reflective paint, and/or other special coatings to help provide barrier capability.

Walls 314 are anchored, sealed, welded or otherwise integral with the facility's floor 316 in some embodiments to form a monolithic structure that substantially reduces penetration of vermin into the facility 202, e.g., to less than one percent, (or two percent, five percent, ten percent, twenty-five percent, or another acceptable value) of the vermin that would be found in a local dirt-floored or open-windowed barn. In some embodiments, fittings 318 are used on any piping 320 or ductwork 322 that would otherwise break the barrier continuity of the shell's impenetrable fabric or other external building material. Suitable fittings 318 may be formed, like the shell 204, from penetration-resistant materials, and may be sealed in place to prevent contamination of the interior 206 by airborne insects or microbes.

A given embodiment may include a hermetically sealed building whose interior 206 is subject to negative, neutral, or positive air pressure, depending on the embodiment and/or the current operational status of the facility. Positive air pressure is envisioned as being the most effective pressurization for keeping unwanted contaminants from entering the facility interior 206. However, neutral air pressure may also be effective in keeping contaminants out, when used in conjunction with filtration and other decontamination measures, for example.

In some embodiments, an airlock 236 is positioned between the interior 206 and the exterior 208 of the hermetically sealed shell. In some embodiments, the facility 202 is designed so that all entry to the interior 206, whether by an animal, human, or any material being brought into the facility interior, must be accomplished by passing through one or more controlled access points. Some examples of controlled access points include airlocks 236, as well as piping 320 and ductwork 322 with fittings 318 and with valves (not shown) for preventing contamination.

In some embodiments, the airlock 236 is designed with screens or other devices to prevent entry into the facility interior by birds, rodents, vermin, and airborne insects. The airlock also allows the removal of livestock from the interior, e.g., when an animal has become sick enough to be isolated from the rest of a herd, when an animal is being culled, or when a cow is temporarily removed for drying out and calving. The airlock 236 shown has a chamber 324 initially filled with ambient external air that enters into the chamber with the items, people, and/or livestock that are being intentionally brought into the facility. The airlock chamber 324 has an outer door 326 and an inner door 328. Prior to opening the inner door 328 of the airlock chamber, the chamber 324 is filled with clean filtered air that replaces the original air, which is exhausted to the exterior 208. The filtered air may be brought into the chamber 324 from the facility's interior 206 and/or may include exterior air which has been filtered.

Alternately, the airlock chamber 324 may be subjected to positive air pressure from the interior 206 as the inner door 328 opens. In some embodiments, a positive air pressure system 240 provides within the hermetically sealed shell 204 an interior air pressure that is greater than an exterior air pressure present at the shell exterior. The air pressure system 240 includes at least fans and the shell itself, and in some embodiments the air pressure system 240 also includes air filters 330 and/or air pressure monitors 332.

Airborne contaminants 226 can be in the form of, or be carried on, pathogens, bacteria, protozoa, fungi, spores, viruses, dust particles, flying insects, and otherwise airborne insects (carried by a breeze or a bird) that are carriers of disease, for example. In some embodiments, all air 230 entering the facility interior has been filtered. Suitable filters 330 include less than highly efficient particulate arrester (HEPA) filters, other HEPA filters, ultra low particulate arrester (ULPA) filters. In some embodiments, air filtration includes use of ozone generators, other forms of oxygenators, exposure to ultraviolet light, and/or irradiation, singly or in combination one with another.

In some embodiments, including some positive air pressure embodiments and some other embodiments, the decontaminable livestock facility's decontamination system 224 includes an air filter 330 which captures at least 80% of airborne particles that are at least 0.3 microns in size. In a variation, an air filter 330 captures at least 70% of airborne particles that are at least 0.3 microns in size. In another variation, an air filter 330 captures at least 95% of airborne particles that are at least 0.3 microns in size. Other percentages and/or particle sizes may be used in other embodiments.

In particular, in one embodiment, the filters 330 will capture at least 98% of all particles in the intake air that are 0.3 microns or larger in size. Such filtration would remove most dust particles, essentially all spores, bacteria, and airborne insects (including flies). However, such filtration would miss many viruses due to their tiny size. In some embodiments, oxygenation of incoming air is used to kill viruses before they enter the facility interior.

In some embodiments, exhaust air from inside the facility interior is also incinerated, re-scrubbed, and/or oxygenated, in addition to being temperature controlled and re-circulated. In some embodiments, air from inside the facility interior is incinerated or otherwise scrubbed or treated without recirculation, to help maintain internal contaminants at a low level. Some embodiments include a burner system 246 for burning airborne contaminants in air fed into the burner system from air within the decontaminable livestock facility. In some embodiments, the burner system is part of an electric power generation system 248.

In addition to filtering/scrubbing all intake air, in some embodiments all exhaust air is also filtered/scrubbed or otherwise treated by the decontamination system 224. Treating exhaust air helps keep pathogens out of the facility's interior, and may also reduce air pollutants 334 and/or odors 336 that would otherwise be emitted from the facility 202. Treatment of exhaust air may significantly reduce greenhouse gases and other forms of pollution that typically emanate from a conventional dairy barn or other conventional livestock facility. Such pollutants 334 may include, for example, pathogens, dust, water vapor, ammonia, volatile organic compounds, methane gas, high levels of carbon dioxide, and similar types of pollutants and/or odor causing substances.

The decontamination system 224 is also sometimes referred to as a “contaminant removal system”. The scope of the decontamination system 224 may be viewed narrowly in terms of devices and procedures whose primary or sole purpose is limited to decontamination, but the decontamination system 224 may also be defined more broadly to also include devices and procedures that serve decontamination as well as other purposes. A narrow view would include filters 330 on incoming air, for example, while a broader view would also include the airlock 236, airtight containers for transporting decontaminated feed into the facility, and filters 330 on outgoing air.

In some embodiments, the contaminant monitoring system 238 includes at least one sensor for sensing within the interior 206 a level of at least one of the following: ammonia, methane, carbon dioxide, airborne particles. In some embodiments, an alarm is sent (via display lights, speakers, email, text message, and/or fax, for example) when a predetermined contaminant level is exceeded. In some embodiments, control signals are sent from the monitoring system 238 to the decontamination system 224 filters and scrubbers to increase decontamination efforts when a predetermined contaminant level is exceeded.

In some embodiments, the facility is tested for contaminants and monitored for at least a predetermined period of time prior to use of the facility for dairy cows or other livestock, to ensure that the interior 206 is decontaminated of undesirable substances prior to initially admitting livestock. Suitable protocols and/or techniques may be used for achieving this state of initial cleanliness, including but not limited to ultraviolet light, irradiation, numerous air volume exchanges of clean filtered air, elevated oxygen levels injected into the facility, sterilization of all interior surfaces, pressure tests, and so on.

In some embodiments, the airlock 236 is equipped with a coded entry system 338 that will only allow authorized personnel to gain entry. Other security measures in some embodiments include a video or web-based camera 340 that is constantly staffed in order to verify that the person seeking entry is authorized and is following required decontamination procedures. The airlock chamber 324 may also utilize compressed air blowers 342 to help decontaminate clothing and other items within the chamber.

In some embodiments, the airlock chamber 324 contains filtered water showers 344 for cleansing animals that are being brought into the facility interior. Separate showers 344 for humans who have recently been in contact with animals in less secure locations may be integrated with access to the airlock.

More generally, the airlock may utilize familiar and hereafter developed decontamination and/or security techniques and/or devices to enhance the bio-security (decontamination) and/or physical security of the facility and its contents.

In some embodiments, all water 232 to be used in the facility 202, whether for washing, livestock consumption, and/or other purposes, is also filtered or otherwise cleansed by a water treatment system 250 to remove as many pathogens as possible before the water reaches the facility interior 206. Water decontamination technologies may be used singly or in combination by the water treatment system, including reverse osmosis, filtration, activated charcoal, ceramic or molecular sieves, and/or concentrated ultraviolet light, for example. Water 232 may also be recovered, recycled, and reused within the facility 202. For example, water from air dehumidification within the facility interior can be used instead of, or in addition to, filtered/treated water brought from outside.

In some embodiments, all livestock feed 234 to be used in the facility 202 is decontaminated to remove pathogens before the feed reaches the facility interior 206. For example, in one embodiment the interior of the hermetically sealed shell encloses at least five kilograms of livestock feed 234 which contains no contaminant 226 at more than 20% of a current action level for that contaminant as established by the United States Food and Drug Administration. Some embodiments use aerobic or anaerobic filters on livestock feed to help provide an agricultural clean room environment.

In some embodiments, a decontaminable livestock facility includes a hydroponic fodder or other feed growth facility 110. Livestock feed 234 is grown in a decontaminated area of the facility 110, and then transported to the livestock 114 in the interior 206 of the hermetically sealed shell through a hermetically sealed tunnel or other passage that provides communication, possibly via airlocks, between the feed growth facility 110 and the main livestock housing in the interior 206.

Some embodiments are automated, with computer-controlled feeding, mechanically-controlled feeding, other remote-controlled feeding, and/or automated/mechanical manure removal, for example, thereby helping further provide an agricultural clean room environment suitable for producing nutraceutical-grade milk. In particular, some embodiments use automation to reduce direct contact between livestock 114 and humans during milking. Some embodiments include robotic milking systems, parallel or herringbone milking parlors, milking rounds or carousels, or another type of automated milking equipment 212. However, if a human milker is properly attired in protective garments, then hand milking of cows may be accomplished without direct contact between human and cow.

Some embodiments use automated cow feeding equipment 214 and/or automated watering equipment 216 to reduce contamination risk. For example, some embodiments use individual feeders and waterers for each animal.

Some embodiments include automated manure removal equipment 218 such as cable-pulled scrapers or water flush systems. In one embodiment an automated manure alley scraping system 218 drops the manure down into pipes that carry it away to be processed. In some embodiments, small diameter water pipes installed near each curb of each alleyway automatically sprinkle a small amount of water onto the alleyway to facilitate removal of the manure while reducing the risk that the undesirable components in manure will volatilize into the air. Water is sprinkled at most six to twelve inches (for example) above the alleyway floor, because wet udders are more vulnerable to disease. Some manure removal systems 218 include a penetration-resistant passageway for the manure (e.g., via buried pipes 320) out of the facility, thereby avoiding passage of manure through the main airlock(s) 236. Valves in the piping reduce contamination of the interior 206. Some embodiments include mobile equipment 218 such as skid-steer loaders or other manure collection devices, which pass through the airlock 236 but are subject to decontamination before entering the interior 206.

Some embodiments include a biologically inert livestock bed 220 situated within the interior 206 of the hermetically sealed shell. In order to reduce human contact with dairy cattle in a facility, for example, some embodiments have a bedding system 220 that does not require significant maintenance and does not produce significant dust. One such bedding system is a coverless single or dual chambered water bed designed for cattle. Some suitable systems generally do not require any daily maintenance, and some also facilitate the automated removal of manure. Such a bedding system would generally be employed in a modern free-stall design, but a variety of bedding and/or stall/pen/accommodation systems within the facility 202 may be used.

Dairy cow lameness can occur from forcing a cow to stand on very hard surfaces such as concrete. Some embodiments build the facility using softer materials such as rubber or flexible plastic for the floor 316, instead of concrete. In some embodiments, a concrete floor is used, but in locations where a cow may be forced to stand the floor 316 includes a covering over the concrete, formed of cushioning and/or softer non-slip materials such as rubber or synthetic rubber. Some embodiments eliminate or minimize use of sand as a bedding material, and some embodiments eliminate or minimize any other bedding material that is known to irritate the cow's hoof, hock, and/or leg and/or aggravate lameness within dairy cattle.

Some embodiments include a temperature control system 242 for controlling temperature within the decontaminable livestock facility, thereby maintaining an ambient temperature suitable for livestock 114 housed within the decontaminable livestock facility interior 206. For example, intake air might be chilled or heated depending on the climate at the specific facility location. Given that a lactating dairy cow is capable of generating approximately fifteen times as much body heat as a human, cooling will often be important. Air cooling can be accomplished with the temperature control system 242 by using ambient air temperature, standard air conditioning equipment, combined heat and power (CHP) subsystems that produce chilled water, naturally occurring chilled water, heat pumps, air-to-air or liquid-to-air heat exchangers, fans, water foggers, misters, sprayers, sprinklers, baths, and so on. Heating the air could be accomplished with the temperature control system 242 by using standard heat exchangers, heat pumps, furnaces, or other measures for warming the intake air.

One specific area of cow comfort is allowing the dairy cow inside the facility to completely avoid heat stress. When a cow is exposed to heat stress, not only does its milk production suffer, but its ability to feed properly, reproduce, and maintain its auto-immune system may all become compromised. Heat stress can be triggered by temperature, humidity, or both. A decontaminable livestock facility may include a temperature control system 242, a humidity control system 244 for maintaining an ambient humidity suitable for livestock housed within the decontaminable livestock facility, or both. Some embodiments of the facility maintain a desirable temperature and humidity level inside, for the livestock and the livestock products involved, regardless of the ambient temperature and humidity outside the facility.

Although heat stress may be avoided by allowing sufficient quantities of filtered, chilled/dried air to enter the facility interior 206, other techniques might also be used. Some embodiments include building designs that allow for indirect heating and cooling of the facility interior by insulation and temperature control of the building structure itself with heating/cooling elements placed inside the building walls 314 and/or roof rather than in the building interior; by circulation of chilled water within or beneath the bedding systems (such as the water beds), within piping, and/or within other structural or non-structural materials within the facility that allow for radiant or other forms of cooling; and/or equipment designed for reduction of humidity from the interior of building spaces. For informational purposes, the optimal temperature for a dairy cow is between 35 and 70 degrees Fahrenheit with relative humidity not in excess of 50%; thermal neutral temperature for a dairy cow is about 66 degrees Fahrenheit. However, not every embodiment need meet these or another optimum standard for temperature or humidity.

Some embodiments include a hermetically sealed dairy barn or other animal-raising building with one or more airlocks. The building may be air-conditioned; it may circulate cold water through piping and/or other mechanisms. Cow waterbeds may sit on cold water pipes. Heat produced by cows may help keep the facility warm. Methane produced by cows may help power air-conditioning. An embodiment may collect methane in pockets (e.g., below a ceiling) in a high-enough concentration to be non-explosive; this methane handling differs from approaches that try to move lots of air through a barn to keep methane concentrations low enough to be non-explosive. Some embodiments seek gentle low airflow rather than high air flow through barn.

Not every item shown in the Figures need be present in every embodiment. Although some possibilities are illustrated here in text and drawings by specific examples, embodiments may depart from these examples. For instance, specific features of an example may be omitted, renamed, grouped differently, repeated, positioned differently, or be a mix of features appearing in two or more of the examples. Functionality shown at one location may also be provided at a different location in some embodiments.

Animal Husbandry Methods

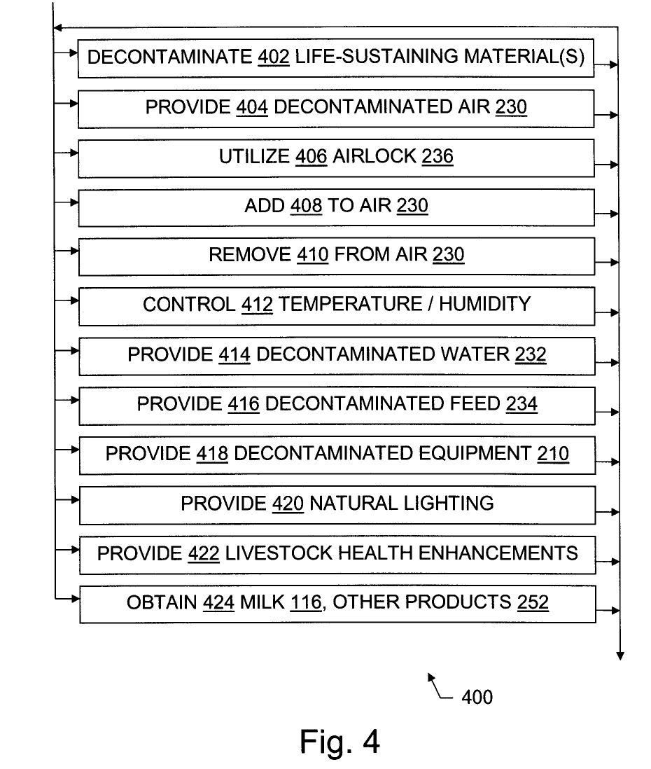

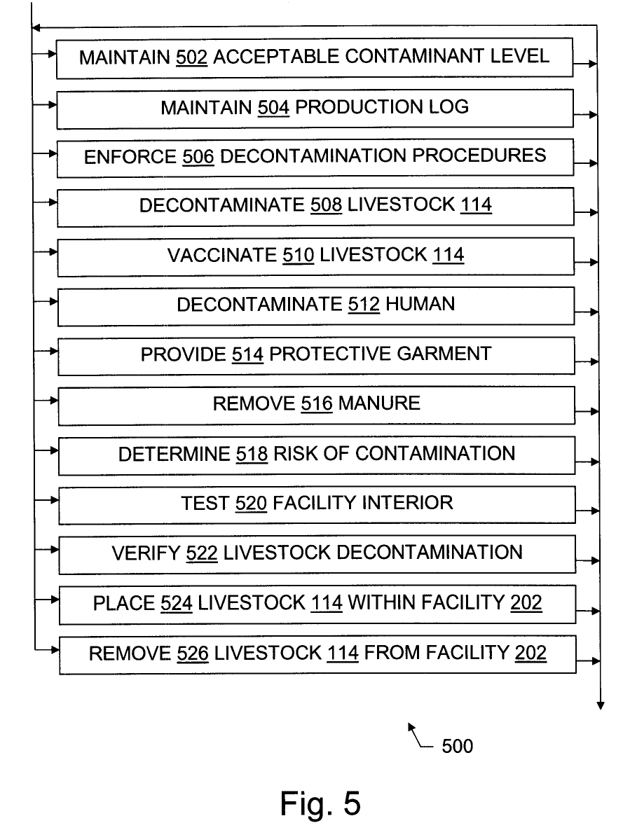

FIG. 4 illustrates method steps in a flowchart 400, and FIG. 5 illustrates method steps in a flowchart 500. These method steps are shown in two figures instead of one figure largely in response to formal requirements for patent drawings. Accordingly, a given method embodiment may include steps from FIG. 4, steps from FIG. 5, or both. In a given embodiment zero or more illustrated steps of a method may be repeated, perhaps with different livestock or contaminants to operate on, for example. Steps in an embodiment may also be done in a different order than the top-to-bottom order that is shown in the Figures. Steps may be performed serially, in a partially overlapping manner, or fully in parallel. The order in which flowcharts 400 and 500 are traversed to indicate the steps performed during a method may vary from one performance of the method to another performance of the method. Flowchart traversal order may also vary from one method embodiment to another method embodiment. Steps may also be omitted, combined, renamed, regrouped, or otherwise depart from the illustrated flowcharts, provided that the method performed is operable and conforms to at least one claim.

Method steps are further described below. All of the illustrated steps are introduced individually. Then additional examples are given, exploring the steps in greater detail, both individually and in relation to one another and in relation to livestock facilities and livestock products.

During a decontaminating step 402, at least one life-sustaining material (air, water, feed) is decontaminated. Decontamination may satisfy a specified absolute contaminant level, a specified relative contaminant level, or a specified decontamination procedure requirement, as described herein.

During a decontaminated air providing step 404, decontaminated air is provided within a livestock facility 202.

During an airlock utilizing step 406, an airlock 236 is utilized to reduce contamination risk while transporting humans, livestock, equipment, manure, life-sustaining materials, and/or other items between the facility interior 206 and the exterior 208.

During an adding step 408, contamination-reducing material such as supplemental ozone and/or supplemental oxygen is added to air within the facility's interior 206.

During a removing step 410, pollutants are removed from air which then either circulates within the facility's interior or is exhausted to the facility's exterior 208. More generally, some embodiments remove 410 an airborne contaminant from a hermetically sealed decontaminated facility through at least one of the following: a filter, an airlock, an airtight container.

During a temperature/humidity controlling step 412, the ambient temperature and/or humidity within the facility's interior is controlled. Step 412 may include modifying temperature, modifying humidity, and/or monitoring temperature and/or humidity within the interior, for example.

During a decontaminated water providing step 414, decontaminated water is provided within a livestock facility 202. For example, water may be treated by at least one of the following decontamination procedures: reverse osmosis, filtration through activated charcoal, filtration through a ceramic sieve, filtration through a molecular sieve, exposure to concentrated ultraviolet light.

During a decontaminated feed providing step 416, decontaminated feed is provided within a livestock facility 202.

During a decontaminated equipment providing step 418, decontaminated livestock equipment 210 is provided within a facility 202.

During a natural light providing step 420, natural light is provided within a livestock facility 202. Natural lighting may be provided, alone or together with artificial lighting, in a controlled agricultural environment (CEA) facility such as a facility 202 for dairy cows or other livestock 114.

During a health enhancements providing step 422, one or more health enhancements such as cushioned flooring, calming music, natural lighting, and exercise equipment 222 are provided to livestock 114 within a facility 202. Other steps taken in some embodiments to provide 422 health enhancements that increase or maximize the health of livestock 114 living within a facility 202 include placing emphasis on excellent nutrition with a high degree of succulent/fresh feed 234, and providing mineral supplements and voluntary herbs, for example. Other examples include providing 422 music (whether intellectually stimulating works such as Mozart, other calming works such as recordings of sounds from pastures, fields, forests, the seashore, running water, etc; or more classical or contemporary music); providing 422 self-actuated grooming brushes or other grooming or massaging devices, providing 422 bovine playground or exercise equipment; providing 422 voluntary milking systems wherein the cow elects how often and when she wishes to be milked, and so on. A methodology, technology, system or technique that improves livestock comfort beyond steps already specified may have a direct bearing on livestock health, and may be employed as part of, or in conjunction with, a given embodiment of the present invention.

During a product obtaining step 424, one or more livestock products 252 such as milk, hormones, or meat, are obtained from livestock which are (or were) housed within a facility 202.

During a contaminant level maintaining step 502, an acceptable low level for at least one contaminant is maintained within a facility interior.

During a production log maintaining step 504, an auditable production log 348 for a facility 202 is maintained, thereby tracking livestock product 252 production and/or tracking actions to reduce or monitor contaminant levels. For example, in one embodiment an auditable production log 348 records access control activity, filtering, and other actions taken to limit contamination. The log 348 may also track contaminant levels of at least one of: the interior 206 of the hermetically sealed decontaminated facility, the milk 116 obtained from the milk-producing animal, the milk-producing animal 114 itself.

During a decontamination enforcing step 506, decontamination procedures for a facility 202 are enforced.

During a livestock decontaminating step 508, at least one livestock 114 animal is decontaminated.

During a livestock vaccinating step 510, at least one livestock 114 animal is vaccinated against an infection or other pathogen.

During a human decontaminating step 512, at least one person is decontaminated. “Person” refers to a human.

During a protective garment providing step 514, at least one person is provided with at least one protective garment. Providing a protective garment to an animal would be considered an example of decontaminating 508 the animal.

During a manure removing step 516, livestock manure is removed from a facility's interior 206. For example, some embodiments remove 516 livestock manure from a hermetically sealed facility 202 through a passageway, such as piping 320, that is not routinely traveled by human personnel. However, in some embodiments manure is removed 526 through an airlock 236.

During a contamination risk determining step 518, a determination is made of a risk of contamination of a facility's interior, e.g., of the risk that an infected animal in the interior 206 will infect other livestock 114.

During a testing step 520, a test is performed to measure or to at least detect a contaminant within a facility's interior 206.

During a decontamination verifying step 522, decontamination of a livestock animal is verified by testing, visual inspection, records review, and/or other measures.

During a placing step 524, a livestock animal is placed within a facility 202, e.g., by being moved from outside the facility to the facility's interior, or by being moved between parts of the facility in which different respective contamination levels are enforced 506 or otherwise maintained 502.

During an animal removing step 526, a livestock animal is removed from within a facility's interior 206.

Process Products Overview

In some embodiments, a method is used to produce a livestock product 252. For example, one embodiment produces uncontaminated milk 116, using a method that includes providing 416 decontaminated feed to a milk-producing animal within a hermetically sealed facility 202; providing 404 decontaminated air to the milk-producing animal within the hermetically sealed facility; providing 414 decontaminated water to the milk-producing animal within the hermetically sealed facility; and then obtaining 424 milk from the milk-producing animal within the hermetically sealed facility.

Acceptable levels of decontamination may be different in different embodiments. In one embodiment, for example, the step of providing 416 decontaminated feed provides feed 234 which contains no contaminant 226 at more than 50% of a current action level for the contaminant established by the United States Food and Drug Administration. In one embodiment, the feed 234 provided 416 contains no contaminant at more than 10% of a current action level for the contaminant established by the United States Food and Drug Administration. Other thresholds below 100% may also be used.

Criteria other than, or in addition to, governmental action levels may also be used to qualify feed. For example, in one embodiment, the provided 416 feed 234 contains no detectable mold. In one embodiment, the decontaminated feed 234 contains less than one percent by weight of foreign matter.

In one embodiment, the feed 234 qualifies because it which was grown in a hermetically sealed facility 110. Decontaminated feed may be grown within the facility 202 itself, or within an adjacent controlled environment building 110 with subsequent transfer of the feed through a connecting sealed passageway into the main facility interior 206.

In a hydroponic fodder facility 110, cereal grains or other suitable seeds such as grass or legumes are sprouted and grown within a controlled environment, and then provided 416 as feed for the cattle or other livestock 114. Seed or other starter material that enters the controlled environment growth facility 110 may be disinfected prior to initiating the sprouting operation, to prevent mold, spores, or bacteria from entering the main facility 202 via the feed. In a variation, plants grown hydroponically but not necessarily in a sealed facility 110 are provided 416 to livestock 114 as feed 234.

While the production of bio-secure milk 116 has been used as an example, it will be understood that some embodiments are configured for other types of livestock 114, such as beef cattle, swine, poultry, sheep, and other animals which are subjects of animal husbandry. Some embodiments can be used for the production of bio-secure meat, eggs, organs, and other animal products 252. Some embodiments produce bio-secure livestock-based foods of types other than milk, or in addition to milk, in facilities 202 for a single animal type such as swine or poultry, while some embodiments include a mixed animal facility 202, such as housing dairy and beef cattle in the same facility.

In some embodiments the livestock 114 are transgenic, with appropriate controls and safeguards in place. In some embodiments pharmaceutical grade or other high grade compositions 252, hormones 252, or other substances are produced in the milk 116 of the livestock.

Some embodiments include a high grade milk 116. For example, in one embodiment a high grade milk 116 is produced by maintaining 502 within a hermetically sealed facility 202 a max-60 contaminant level for each of at least three of the following pathogens 226: tuberculosis, brucellosis, Johne's disease, salmonella, foot-in-mouth disease, contagious mastitis. A “max-60 contaminant level” for a pathogen is less than 60% of a level at which the pathogen would typically occur in milk production absent the maintaining step 502. By milking, one then obtains 424 the high grade milk from the cow or other milk-producing animal within the hermetically sealed facility. In a variation, the maintaining step 502 maintains a max-60 contaminant level for each of at least five of the pathogens. In another variation, the maintaining step 502 maintains a max-20 contaminant level for each of at least four of the pathogens, namely, a level less than 20% of a level at which the pathogen would typically occur in milk production absent the maintaining step.

### ADDITIONAL EXAMPLES

Additional example embodiments are described below. Note that a given embodiment that includes animal husbandry method steps will possibly but not necessarily employ a livestock facility discussed as an example herein. Likewise, a given embodiment may include a livestock facility which is possibly but not necessarily utilized according to animal husbandry method steps discussed as examples herein. A given embodiment may also include milk and/or another livestock product produced using animal husbandry methods steps and/or using livestock facilities discussed as examples herein.

Some embodiments include a method of maintaining a decontaminable livestock facility, including the steps of providing 404 decontaminated air 230 to livestock animals 114 within a hermetically sealed facility 202, and enforcing 506 decontamination procedures on human personnel 302 prior to their entry within the hermetically sealed facility. For example, enforcement 506 may require decontamination procedures such as one or more of the following, to limit contamination of livestock animals by human-animal contact: decontaminating 512 a human before the human enters the hermetically sealed facility 202; providing 514 a human with a protective garment 346 to be worn within the hermetically sealed facility.

In particular, enforcement 506 may require providing a human 302 with a sterile suit as a protective garment 346 to be worn within the hermetically sealed facility. The sterile suit, like other protective garments 346, should be adequate to maintain the decontamination integrity of the facility 202 but need not always suffice for other uses such as surgery or semiconductor fabrication.

Some embodiments include steps and systems to reduce contagious mastitis by having the decontamination system 224 filter out and kill pathogens. Other embodiments reduce environmental mastitis by providing 418 water beds 220 or other non-bacteria-hosting materials in locations that may come into contact with the udder and/or teats of a cow. Some embodiments provide 422 proper and frequent sanitization of any milking equipment 212 or milk containers that may be a source for spreading pathogens within milk from a mastitic teat or udder. Some embodiments provide 422, 516 frequent and effectively complete removal of manure and urine from all common areas within the facility interior 202, to prevent other cows from coming into contact with contaminants from an infected cow. Some embodiments provide blowers, humidity controls, disposable towels, and/or use non-absorbent materials to help keep interior 206 surfaces dry to prevent the growth of any undesirable bacteria which may cause mastitis. Other techniques for minimizing mastitis may also be provided 422.

Some embodiments provide 422 holistic nutritional and cow health practices to assist in keeping dairy cows healthy, thereby reducing the occurrence of undesirable pathogens or high somatic cell counts in produced milk 116. Any protocol, technique, technology, or substance that enhances the cow's natural immune system while housed within the facility 202 may be provided 422 in a given embodiment.

While many nutritional elements may be incorporated successfully into a given embodiment, in some embodiments fresh feed 234 is provided 416 instead of dry forage. In addition, reduced use of grain as a feed concentrate is provided 422 to allow better operation of the cow's rumen. Nutritional practices that successfully optimize the functioning of the dairy cow's rumen while providing optimal nutrient, energy, protein, macronutrients and/or micronutrients to the dairy cow living inside the facility can be provided 422. Employment of the hydroponic fodder operation, with feed 234 provided 416 from the sealed feed growth facility 110, may eliminate/minimize livestock 114 exposure to pesticides and insecticides. Dairy cow veterinarian/dairyman examination and/or livestock care systems that minimize the need for antibiotics are also provided 422 within some embodiments. When antibiotics are required, control protocols are provided 422 ensure that the correct antibiotic is given to the correct cow, and an embodiment may also maintain 504 an auditable log pertaining to the use and source of each antibiotic.

According to some embodiments, only cows that are initially healthy and have been vaccinated 510 for tuberculosis, brucellosis, and any other diseases for which effective vaccines exist will be admitted into a herd housed in a facility 202. Acceptable enforcement 506 protocols and procedures are followed to prevent the entry of any animal that is not verified 522 disease free.

For example, some embodiments include testing 520 at least once per month for at least three months for the presence within a hermetically sealed facility 202 of at least three of the following pathogens: tuberculosis, brucellosis, Johne's disease, salmonella, listeria, campylobacter, foot-in-mouth disease, contagious mastitis. The same three or more pathogens are not necessarily the subject on each test occasion.

Some embodiments include verifying 522 that a livestock animal has no detectable levels of at least the following pathogens: tuberculosis, brucellosis. Then the verified livestock animal is placed 524 within an interior 206 room of the hermetically sealed facility 202. Verifying 522 may be accomplished by having a veterinarian examine the animal, by testing for the presence of identifiable pathogens, by examining vaccination records, by quarantining the animal for a specified period of time, and/or by other measures prior to entry of the animal into the facility interior 206.

Some embodiments include vaccinating 510 a livestock animal against a respiratory illness. Then the vaccinated livestock animal is placed 524 within an interior 206 room of the hermetically sealed facility 202.

Some embodiments provide 416 decontaminated feed 234 to livestock 114 within a hermetically sealed facility 202. For example, one embodiment provides livestock feed 234 which contains no contaminant 226 at more than 30% of a current action level for the contaminant established by the United States Food and Drug Administration. Another embodiment provides livestock feed 234 which contains no contaminant at more than 2% of a current action level for the contaminant established by the United States Food and Drug Administration. These are merely examples. Other embodiments set different thresholds, focus on particular contaminants regardless of action levels for other contaminants, and/or refer to action levels set by governmental agricultural authorities other than the United States Food and Drug Administration.

Feed commonly fed to dairy cattle in conventional circumstances is capable of transporting and/or containing many types of pathogens and other undesirable contaminants. Consequently, in order to help assure the bio-security and purity of the milk supply, feed entering the facility 202 is first decontaminated. Decontaminated feed 234 may be provided 416 in conjunction with procedures such as feed sterilization, disinfection, chemical or water rinses, elimination of oxygen from the feed storage areas, increased oxygenation of the feed storage areas, ultraviolet light, irradiation, and/or other decontamination technology.

Some embodiments produce uncontaminated livestock products 252, such as milk, meat, organs, eggs, and/or hormones from livestock. For example, some products 252 are produced by a method that includes utilizing 406 an airlock 236 to limit contamination of an interior 206 of a hermetically sealed facility 202, and testing 520 for the presence of contamination 226 within the hermetically sealed facility. Livestock products 252 are then obtained 424, for human or animal use, from a livestock animal which was housed or presently is housed within the interior 206 of the hermetically sealed facility 202.

Animals may be housed within a facility 202 to prevent contamination while their biological processes formulate or refine the desired product 252. Then, depending on the nature of the product and the harvesting requirements, the product may be obtained inside the facility or elsewhere. Milking can be done within a facility 202, for example, while slaughter may be better performed elsewhere after transporting the animal in a manner that will not excessively contaminate the product 252.

Some products 252 are produced by providing decontaminated life-sustaining materials 228 to livestock 114 ina hermetically sealed facility 202. For example, this may include providing 416 feed 234 which has a lower level of microbes than would be present absent the hermetically sealed facility; providing 404 air 230 which has a lower level of microbes than would be present absent the hermetically sealed facility; and/or providing 414 water 232 which has a lower level of microbes than would be present absent the hermetically sealed facility.

Since humans are a possible source or carrier of cow disease or other contamination 226, some embodiments reduce or minimize any close contact between livestock (e.g., dairy cows) and any human 302 who is not appropriately attired. Some embodiments automate functions inside the facility 202 to reduce the chance for inadvertent contact between humans and dairy cows. Depending on the planned duration, interaction with the cow(s), specific activity, and whether the human has been in contact with any other cows from any other dairies within the recent past or has recently been sick, the human may be required to undergo decontaminating 512 steps such as showering on site, and/or wearing protective garments 346 (e.g., face masks, gloves, appropriate hair/head covering, special boots). In some cases the human may wear a sterile suit that completely encloses the human and thus prevents direct contact between human and cow.

Monitoring devices may be incorporated into the interior of the facility to help enforce 506 strict adherence to decontamination protocols, including closed circuit video and/or web-based cameras 340, and automated air testing 520 devices. The internal conditions of the dairy facility 202 can be monitored as part of maintaining 502 an acceptably low contamination level. Monitoring may track conditions on a continuous 24/7 basis. Monitoring equipment 238, 332, 338, 340 can be integrated into the audit path to help maintain 504 a production log 348 that demonstrates bio-security on a real time or delayed basis.

Some embodiments provide 420 natural lighting to milk-producing animals and/or other livestock 114 within a hermetically sealed facility 202. Direct sunlight is beneficial to a dairy cow's health and the quality of its milk. Some embodiments use building materials in the shell 204, such as windows, skylights, clear or translucent external fabrics, or the like, that allow sunlight to enter the interior 206 of the facility. Some embodiments include a temperature control system 242 and/or a humidity control system 244 which allows substantial sunlight while also providing climate and comfort control of temperature and/or humidity. However, artificial lighting is used in some embodiments. Artificial lighting may also be used in conjunction with natural lighting as a way of extending a dairy cow's exposure to light.

Some embodiments control air quality inside a facility 202 by one or more of the following measures: a temperature control system 242, exclusion of disease vectors by filtering 330 and/or vaccination 510; creation and maintenance of dry conditions inside the facility to prevent growth of harmful bacteria and other contaminants; scrubbing inside the barn to remove 410 carbon dioxide and other gases; adding 408 supplemental oxygen to reduce viral and bacterial growth and/or to replace some of the oxygen used by animals in their respiration; partial or selective removal 410 from the barn of a stream of concentrated gases such as carbon dioxide, methane, and volatile organic compounds.

In some embodiments, conditioning the internal air also controls dust and other particulate contaminants 226. Most dust will originate from either bedding material or dried manure. Hence, some embodiments use bedding materials 220 that are non-dust generating, and some use techniques for not only removing 516 manure, but for doing so in such a way as to minimize dust generation, as well as ammonia and methane generation.

Some embodiments include a method of producing uncontaminated milk 116. One embodiment, for example, includes utilizing 406 an airlock 236 to limit contamination of an interior 206 of a hermetically sealed decontaminated facility 202, and then obtaining 424 milk 116 from a milk-producing animal 114 within the hermetically sealed decontaminated facility 202.

Some embodiments also include at least one of the following steps to limit contamination from human-animal contact: decontaminating 512 a human 302 before the human enters the hermetically sealed decontaminated facility 202, and providing 514 the same or another human 302 with a protective garment 346 to be worn within the hermetically sealed decontaminated facility 202.

In some embodiments, a hermetically sealed decontaminated facility 202 encloses multiple milk-producing animals. In such embodiments, it may be determined 518 by medical testing, statistical prognoses, herd size, product purity requirements, and/or other factors that a first milk-producing animal within the hermetically sealed decontaminated facility 202 poses an unacceptable risk of contamination to another milk-producing animal within the hermetically sealed decontaminated facility 202. In that event, the first milk-producing animal may be removed 526 from the hermetically sealed decontaminated facility 202, through an airlock 236 or otherwise. Alternately, the milk-producing animal may be decontaminated 508 and/or vaccinated 510 in place. In general, livestock 114 will also be decontaminated 508 before being initially placed 524 in a hermetically sealed decontaminated facility. In some cases, livestock 114 will also be vaccinated 510 before being initially placed 524 in a hermetically sealed decontaminated facility.

Some embodiments prevent any external contaminant 226 from gaining entry sufficiently to compromise the desired bio-secure nature of the facility 202. However, as a result it will also be the case that internally-generated contaminants 226 such as ammonia, methane, carbon dioxide, animal dander, dust, dried manure particles, volatile organic compounds, and odors will not be able to readily escape without assistance. Therefore some facilities 202 incorporate features that allow these contaminants to be monitored within the facility and eliminated in a controlled fashion.

Some embodiments use ongoing monitoring by devices, whether chemical, physical, or visual, for monitoring the purity of the internal air, water, and contact surfaces. For example, in some embodiments air monitors 238 activate filtered intake/exhaust ventilation systems as needed. Contaminant threshold detection may also cause actions such as absorption and/or adsorption of undesirable contaminants from the interior air by filters 330; incineration by burners 246; separation; chemical reactions such as adding 408 substances to neutralize the contaminant(s); and filtering out dust and dander from the internal air. Some techniques utilize the relatively low turbulence of the air within the facility itself for separation, isolation, destruction, neutralization, or capture of undesirable substances within the air, as discussed herein with regard to methane, for example. Each of the foregoing actions may be considered part of the step of providing 404 decontaminated air (or providing 414 decontaminated water, in some cases) in a given embodiment. Such actions may be performed within or without the facility interior 206.

In particular, some embodiments selectively remove 410 from air within the hermetically sealed facility at least one of the following: a volatile organic compound as defined by a governmental regulatory agency, methane, ammonia, carbon dioxide. In some embodiments, exhaust air from a sealed barn or other facility 202 is treated if necessary (e.g., by a water scrub), and then used as input air for a gas-fired turbine or other burner in an electric power generation system 248. Volatile organic compounds and methane, both of which will be dilute, will be incinerated as fuels in the process of generating electricity. Manure may also be converted into usable products, such as fertilizer, or methane gas to fire a gas turbine. The final oxidized forms of volatile organic compounds and methane will be mainly carbon dioxide.

The separation, isolation, or special handling of methane gas that the dairy cows emit is part of some embodiments. Methane gas is explosive and/or flammable when mixed with air in a percentage between 5% and 15% methane. In a hermetically sealed facility, it may be important to manage the concentration of methane gas within the facility, e.g., by employing a contaminant monitoring system 238 and a decontamination system 224 for methane management. In some embodiments, the systems are designed for dilution of methane to below 5%. In some embodiments, the systems are designed for addition of extra methane gas to ensure a concentration in excess of 15%. Some facilities limit permissible electronics and electrical equipment to prevent methane ignition. Techniques and/or substances may also be administered to dairy cows in order to reduce the amount of methane emission.

### CONCLUSION

In short, some embodiments center on providing a dairy cow with a protected, ultra-low pathogen/contaminant facility that is also otherwise designed for the cow's comfort and health, to produce bio-secure milk while keeping the dairy cow healthy and minimizing any pathogenic or other contamination in the milk produced by the dairy cow. Some embodiments use a device, technique, technology, protocol or procedure that allows a dairy cow to reside comfortably within the confines of a hermetically sealed facility consuming sterilized or otherwise decontaminated air, water, and food. Some embodiments provide a method for producing cow's milk that is very pure and biologically secure, herein referred to as bio-secure milk. Such a method may help eliminate or minimize disease or contamination from the cow and the environment in which the cow lives. Some embodiments provide the cow with superior nutrition and comfort within a controlled environment. The incidence of disease, poisoning, or other forms of contamination in the cow and its milk can be extremely low, whether measured directly as harmful bacteriological substances (such as pathogens that can cause Tuberculosis, Brucellosis, Johne's disease, Salmonella, Foot-in-mouth (Mad Cow) disease, Contagious Mastitis, etc) and/or measured by indirect indicators of sub-acute or acute infection (somatic—or white—cell count is often used as such an indicator). Such indicia found in the milk produced by the cows will be measurably lower than normal industry standards, thus assuring a high grade and purity of the milk produced that is demonstrably bio-secure. Some embodiments provide an auditable chain of production which demonstrates the bio-security of the milk. It may also be possible with some embodiments to produce a milk of exceptional quality without actually achieving a level adequate to be considered bio-secure milk. Any such milk produced by a method disclosed herein, whether bio-secure or not, may be part of an embodiment.

Not every embodiment provides full bio-security for livestock; simple improvements over contamination levels in conventional facilities may suffice; partial implementations are implementations nonetheless. For example, one may employ bio-security tools and techniques less stringently, and not achieve the highest degree of bio-security, health, and milk purity envisioned here within, while nonetheless still embodying the invention. Although a facility 202 may employ or be subject to clean room style technologies, it will not necessarily be clean in a lay sense. Living animals generate manure, lose dander, respire, and otherwise produce contaminants. Assume that an embodiment controls contaminant levels in a facility's interior 206 and reduces or minimizes entry of external contaminants by decontaminating air, water, and/or food; prevents humans who enter the facility from bringing in diseases by using automated milking, protective garments, and access controls such as an airlock; prevents animals from bringing in disease by vaccination and quarantine; and utilizes protocols and technologies inside the facility 202 to prevent any disease contained within one or more animals from being able to grow and spread by contact with diseased animals or their manure. Such an embodiment, or even an embodiment that does only some of these things, nonetheless provides a facility 202 and uncontaminated livestock products 252. An embodiment need not have the visual appearance or all the other characteristics of a “clean” room as that term would be used in lay non-agricultural contexts, or in semiconductor foundries or surgical theatres.

An embodiment may employ aspects discussed here with some additional techniques, protocols, procedures, technology, etc. singly or in combination that are not specifically mentioned herein, but which are consistent with a purpose stated herein. For example, different numeric thresholds could be specified; different pathogens could be controlled; and/or steps listed could be combined in different ways.

Although particular embodiments of the present invention are expressly illustrated and described herein as methods, for instance, it will be appreciated that discussion of one type of embodiment also generally extends to other embodiment types. For instance, the descriptions of methods also help describe facilities and their component systems, and help describe products (such as pure raw milk, or a decontaminated cow) that are produced by methods. It does not follow that limitations from one embodiment are necessarily read into another.

Reference has been made to the figures throughout by reference numerals. Any apparent inconsistencies in the phrasing associated with a given reference numeral, in the figures or in the text, should be understood as simply broadening the scope of what is referenced by that numeral.

As used herein, terms such as “a” and “the” are inclusive of one or more of the indicated item or step. In particular, in the claims a reference to an item generally means at least one such item is present and a reference to a step means at least one instance of the step is performed.

Headings are for convenience only; information on a given topic may be found outside the section whose heading indicates that topic.

All claims as filed are part of the specification.

While exemplary embodiments have been shown in the drawings and described above, it will be apparent to those of ordinary skill in the art that numerous modifications can be made without departing from the principles and concepts set forth in the claims. Although the subject matter is described in language specific to structural features and/or methodological acts, it is to be understood that the subject matter defined in the appended claims is not necessarily limited to the specific features or acts described above the claims. It is not necessary for every means or aspect identified in a given definition or example to be present or to be utilized in every embodiment. Rather, the specific features and acts described are disclosed as examples for consideration when implementing the claims.

All changes which come within the meaning and range of equivalency of the claims are to be embraced within their scope to the full extent permitted by law.

What is claimed is:

1. A method of producing uncontaminated milk, the method comprising the steps of:
utilizing an airlock to limit contamination of an interior of a hermetically sealed decontaminated facility; and
obtaining milk from a milk-producing animal within the hermetically sealed decontaminated facility.

2. The method of claim 1, further comprising at least one of the following steps to limit contamination from human-animal contact:
decontaminating a human before the human enters the hermetically sealed decontaminated facility;
providing a human with a protective garment to be worn within the hermetically sealed decontaminated facility;
providing a human with a sterile suit to be worn within the hermetically sealed decontaminated facility.

3. The method of claim 1, wherein the hermetically sealed decontaminated facility encloses multiple milk-producing animals, and the method further comprises the steps of:
determining that a first milk-producing animal within the hermetically sealed decontaminated facility poses an unacceptable risk of contamination to another milk-producing animal within the hermetically sealed decontaminated facility; and
through the airlock, removing the first milk-producing animal from the hermetically sealed decontaminated facility.

4. The method of claim 1, further comprising the steps of:
decontaminating the milk-producing animal; and then
placing the decontaminated milk-producing animal in the hermetically sealed decontaminated facility.

5. The method of claim 1, further comprising the steps of:
vaccinating the milk-producing animal; and then
placing the vaccinated milk-producing animal in the hermetically sealed decontaminated facility.

6. The method of claim 1, further comprising the step of providing decontaminated feed to the milk-producing animal within the hermetically sealed decontaminated facility.

7. The method of claim 1, further comprising the step of providing decontaminated air to the milk-producing animal within the hermetically sealed decontaminated facility.

8. The method of claim 1, further comprising the step of adding at least one of the following to air within the hermetically sealed decontaminated facility: supplemental oxygen, supplemental ozone.

9. The method of claim 1, further comprising the step of selectively removing from air obtained from within the hermetically sealed decontaminated facility at least a portion of at least one of the following: a volatile organic compound as defined by a governmental regulatory agency, methane, ammonia, carbon dioxide.

10. The method of claim 1, further comprising the step of providing decontaminated water to the milk-producing animal within the hermetically sealed decontaminated facility.

11. The method of claim 1, further comprising the step of providing natural lighting to the milk-producing animal within the hermetically sealed decontaminated facility.

12. The method of claim 1, further comprising the step of providing decontaminated equipment within the hermetically sealed decontaminated facility.

13. The method of claim 1, further comprising the step of maintaining an auditable production log which records actions taken to limit contamination and which also tracks contaminant levels of at least one of: the interior of the hermetically sealed decontaminated facility, the milk obtained from the milk-producing animal, the milk-producing animal itself.

14. The method of claim 1, further comprising the step of removing manure of the milk-producing animal from the hermetically sealed decontaminated facility through the airlock.

15. The method of claim 1, further comprising the step of removing an airborne contaminant from the hermetically sealed decontaminated facility through at least one of the following: a filter, an airlock, an airtight container.

16. A method of producing uncontaminated milk, the method comprising the steps of:
providing decontaminated feed to a milk-producing animal within a hermetically sealed facility;
providing decontaminated air to the milk-producing animal within the hermetically sealed facility;
providing decontaminated water to the milk-producing animal within the hermetically sealed facility; and
obtaining milk from the milk-producing animal within the hermetically sealed facility.

17. The method of claim 16, wherein the step of providing decontaminated feed provides feed which contains no contaminant at more than 50% of a current action level for the contaminant established by the United States Food and Drug Administration.

18. The method of claim 16, wherein the step of providing decontaminated feed provides feed which contains no contaminant at more than 10% of a current action level for the contaminant established by the United States Food and Drug Administration.

19. The method of claim 16, wherein the step of providing decontaminated feed provides feed which contains no detectable mold.

20. The method of claim 16, wherein the step of providing decontaminated feed provides feed which contains less than one percent by weight of foreign matter.

21. The method of claim 16, wherein the step of providing decontaminated feed provides feed which was grown in a hermetically sealed facility.

22. The method of claim 16, wherein the step of providing decontaminated air provides air through a filter which captures at least 70% of airborne particles that are at least 0.3 microns in size.

23. The method of claim 16, wherein the step of providing decontaminated air provides air through a filter which captures at least 95% of airborne particles that are at least 0.3 microns in size.

24. The method of claim 16, wherein the step of providing decontaminated water provides water which has been treated by at least one of the following decontamination procedures: reverse osmosis, filtration through activated charcoal, filtration through a ceramic sieve, filtration through a molecular sieve, exposure to concentrated ultraviolet light.

25. A high grade milk produced by a process comprising the steps of:
maintaining within a hermetically sealed facility a max-60 contaminant level for each of at least three of the following pathogens: tuberculosis, brucellosis, Johne's disease, salmonella, foot-in-mouth disease, contagious mastitis;
wherein a max-60 contaminant level for a pathogen is less than 60% of a level at which the pathogen would typically occur in milk production absent the maintaining step; and
obtaining the high grade milk from the milk-producing animal within the hermetically sealed facility.

26. The high grade milk of claim 25, wherein the maintaining step maintains a max-60 contaminant level for each of at least five of the pathogens.

27. The high grade milk of claim 25, wherein the maintaining step maintains a max-20 contaminant level for each of at least four of the pathogens, namely, a level less than 20% of a level at which the pathogen would typically occur in milk production absent the maintaining step.

28. The high grade milk of claim 25, wherein the obtaining step obtains the high grade milk from a cow within the hermetically sealed facility.
